(19) United States
(12) Patent Application Publication
(10) Pub. No.: US 2006/0110418 A1
(43) Pub. Date: May 25, 2006

(54) Water-in-oil emulsions and methods

(75) Inventor: Keith H. Johnson, Cambridge, MA (US)

Correspondence Address: Eugene Berman;Law Offices of Eugene Berman, 26 Cedarwood Court, Rockville, MD, 20852, US

(73) Assignee: Nanocluster Technologies, LLC

(21) Appl. No.: 11/285,878

(22) Filed: Nov. 23, 2005

Related U.S. Application Data
(63) Continuation-in-part of application No. 09/662,195, filed on Sep. 14, 2000.

(51) Int. Cl. A61K 8/42; A61K 8/06

(52) U.S. Cl. 424/401; 977/926

(57) ABSTRACT

The present invention provides a process for delivery of water nanoclusters of diameter less than about one nanometer to the skin to yield high epidermal permeability and improved delivery of water to within the outer layer of human skin. This invention provides effective water-cluster-based formulations for a broad range of stable water/oil nanoemulsion configurations.

### BACKGROUND OF THE INVENTION

Much of the cosmetic industry has been and continues to be focused on the development of effective skin moisturizers to help overcome the skin hydration barrier. However, the typical cosmetic moisturizing formulation uses oil formulations to deliver various active ingredients, with water present as a non-active ingredient carrier, which typically evaporates from the skin surface

The pharmaceutical industry has likewise devoted a significant part of its resources toward the development of drugs that can be delivered transdermally for the treatment of afflictions ranging from skin disorders to bodily disease. Transdermal drug delivery systems provide for the controlled release of drugs directly into the bloodstream through intact skin. Transdermal drug delivery is an attractive alternative that can be used often when oral drug treatment is not possible or desirable. In particular, with transdermal administration long duration of action and controlled activity is achieved.

Industry is continually seeking to develop more effective applications of beneficial formulations to the skin.

### BRIEF SUMMARY OF THE INVENTION

The present invention provides water nanocluster/oil (W/O) formulations and methods for delivering water nanoclusters to the skin. In one aspect, the invention provides a process for the delivery of water nanoclusters through the outermost layer of human skin by preparing a water nanocluster composition comprising water nanoclusters having a least one dimension between about 0.5 and 10.0 nanometers (about 5-100 Angstroms) and an oil formulation as a W/O emulsion, and applying said water nanocluster composition onto the outermost layer of human skin.

The present invention also provides a water nanocluster/oil W/O emulsion composition comprised of (1) about 5 to 50% by weight water containing water nanoclusters having at least one dimension between about 0.5 and 10.0 nanometers (about 5-100 Angstroms), and preferably less than about 1.0 nanometer, (2) about 5 to 50% by weight of one or more surfactants selected from the group consisting of fatty acids, ethoxylates and alcohols, and (3) about 10 to 90% by weight being oils, including other beneficial ingredients.

### BRIEF DESCRIPTION OF THE DRAWINGS

FIG. 1 depicts a pentagonal 5-molecule water nanocluster

FIG. 2 depicts a 20-molecule pentagonal dodecahedral water nanocluster

FIG. 3 depicts a 20-molecule pentagonal dodecahedral water nanocluster interacting with a typical fatty acid surfactant, oleic acid. The red spheres represent oxygen atoms, the blue spheres represent carbon atoms, and the white spheres represent hydrogen atoms.

FIG. 4 depicts the ability of the cage structure of the water nanocluster to engulf and clathrate the hydrophobic lipid molecule to counteract the hydrophobic effects of the lipid hydrophobes.

FIG. 5 depicts the ability of the outermost electronic structure of the water nanocluster to give up an electron and function as an antioxidant.

FIG. 6 depicts the ability of the outermost electronic structure of Vitamin E to give up an electron and function as an antioxidant.

FIG. 7 depicts a needle-like array of five pentagonal dodecahedral water clusters sharing a pentagonal face between neighboring dodecahedra.

FIG. 8 depicts an “end-on” view of the needle-like array of water clusters shown in the above FIG. 7. Note the cavity that runs down the length of the needle.

FIG. 9 depicts the ability of the outermost electronic structure of the needle-like array of water clusters shown in FIG. 7 to give up an electron and function as antioxidant. (Cf. FIG. 5).

FIG. 10 depicts the stabilization of the needle-like array of water clusters shown in FIGS. 7 by a single fatty-acid surfactant such as oleic acid.

### DETAILED DESCRIPTION OF THE INVENTION

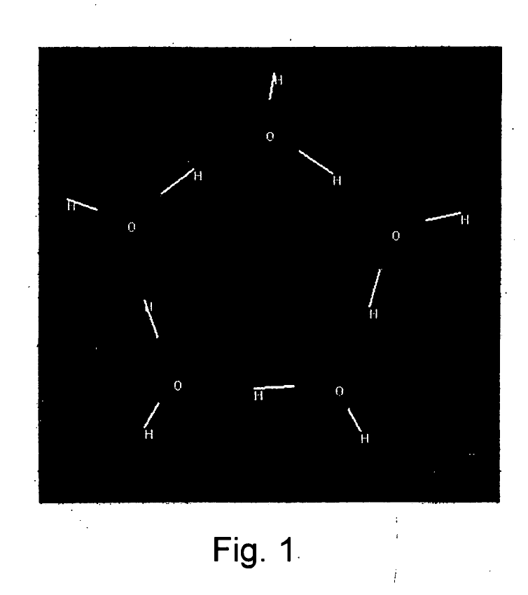

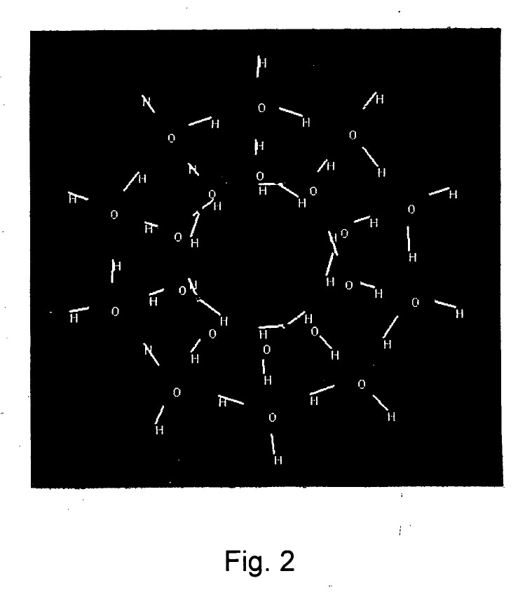

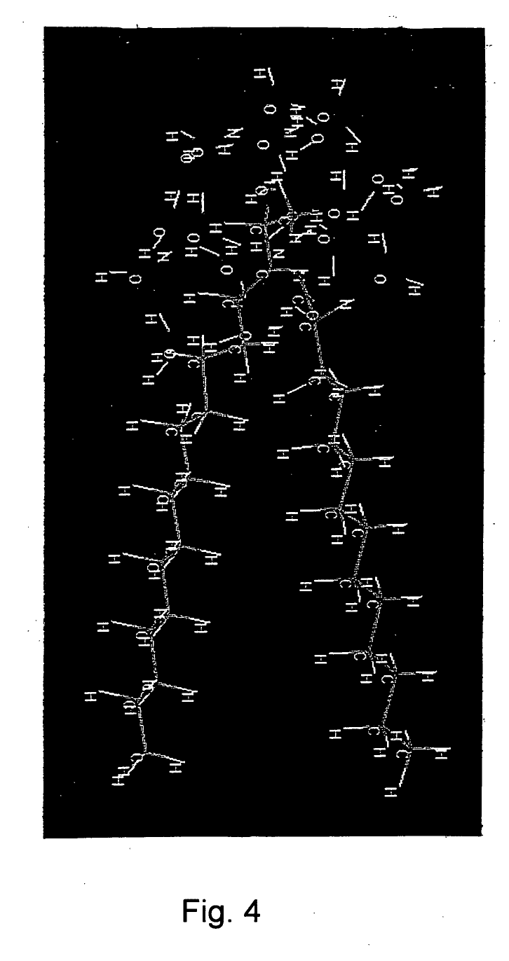

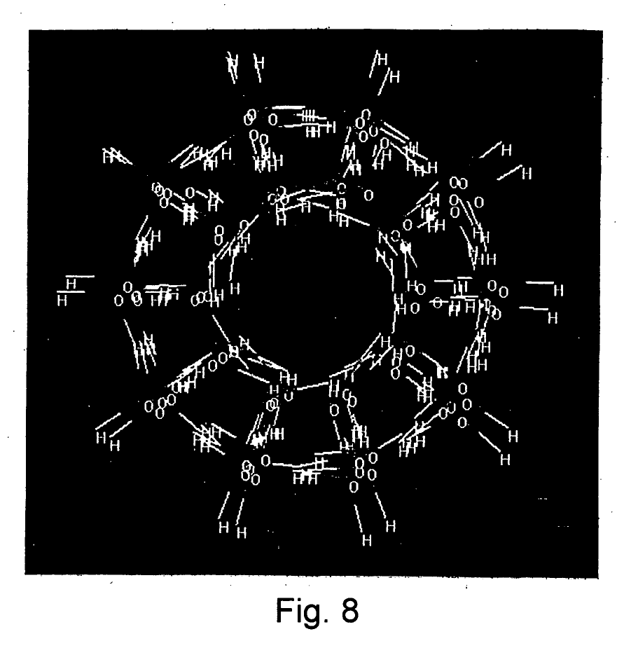

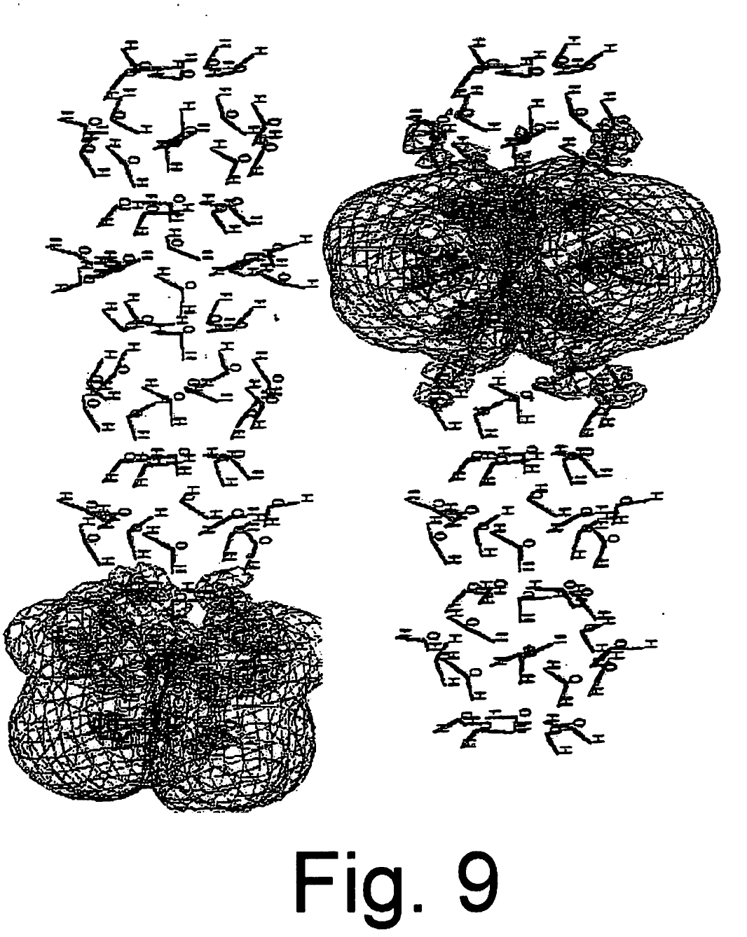

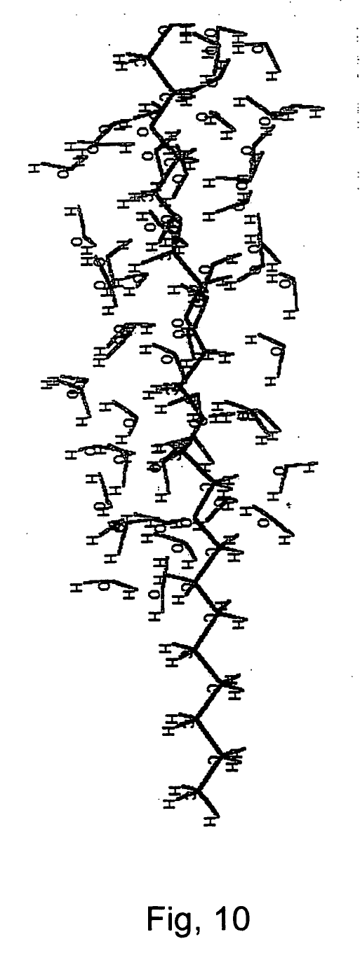

Water clusters of the type used in the present invention are described in U.S. Pat. Nos. 5,800,576 and 5,997,590, both of which are incorporated herein by reference. The specific formulations described therein are waterclusters/fuel emulsions, but the teaching of the form of the water cluster components (e.g., see columns 1-14 and FIGS. 1-10 of U.S. Pat. No. 5,997,590) are the same as those water clusters useful in this invention. The water clusters are preferably concatenated pentagonal water clusters like that shown in FIG. 12 of U.S. Pat. No. 5,800,576 and are comprised of twenty-one or fewer water molecules and having at least one dimension of 8A (0.8 nm) or less. For example, individual water clusters in dodecahedral form are essentially spherical in shape and have a diameter of about 0.8 nanometer (see FIG. 2); those in pentagonal form are puckered rings and have a diameter of about 0.5 nanometer (see FIG. 1).

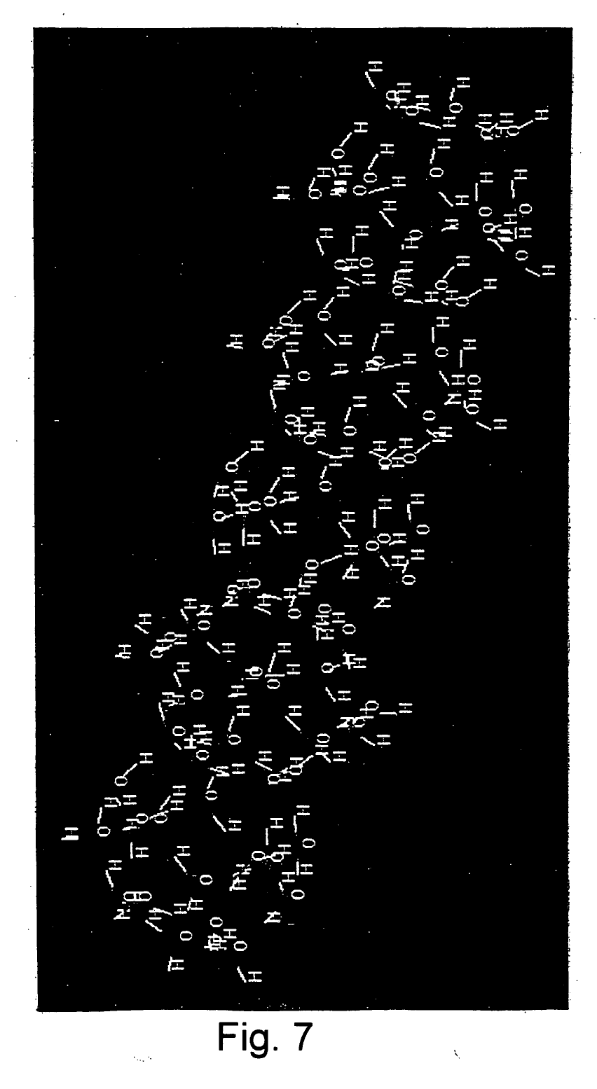

The water clusters can be present as individual water cluster units and/or as an array of aggregated water cluster units. The pentagonal water cluster shown FIG. 2 and the dodecahedral water cluster shown in FIG. 1 are examples of individual water clusters. FIG. 7 shows an array of five dodecahedral water clusters in a needle-like array. One dimension of the array of water clusters is less than about one nanometer (10 Angstroms), with the length of the array being about 3 nanometers (about 30 Angstroms) (see FIG. 7).

The type and size of the individual water clusters, as well as the degree and type of aggregation thereof, will and may vary in a given water cluster formulation of this invention. For example, a given composition of this invention may contain individual pentagonal and pentagonal dodecahedral water clusters, some of which may be in the form of multi-cluster arrays, e.g., needle-like arrays like that shown in FIG. 7. Regardless of the water cluster type, size and degree of aggregation, one dimension of the water cluster or array thereof, about 10.0 nanometers (100 Angstroms), preferably less than about one nanometer (10 Angstroms), most preferably less than or equal to about 0.8 nanometer (8 Angstroms).

All of the water which is present need not be in the form of water clusters. Some of the water may be present in traditional bulk water form (i.e., in the form of globules larger than 10 nanometers or 100 Angstroms in diameter, which exhibit all the physical characteristics of bulk water). Since the benefits of the present invention are attributed to the presence of the water clusters, it is preferred that a substantial (most preferably greater than 50%) portion of the water present be in the water cluster form.

The water nanoclusters of the present invention can be produced by a variety of means as taught in the aforesaid referenced patents (e.g., see columns 9-10 of U.S. Pat. No. 5,997,590). However, for purposes of this invention, use of surfactants to produce the desired nanoemulsion (as described below) is most preferred.

The oil formulations useful herein include for cosmetic applications: cosmetic industry oils such as soybean, peanut, olive, sesame and paraffin. Suitable cosmetic oil formulation may also include any of a variety of additives useful or non-deleterious in a cosmetic product, such as oil soluble vitamins and other cosmetic nutrients (e.g., Vitamin E), fragrances and other active (e.g., sunscreens) or inert additives, which are preferably soluble in the oil.

The preferred oil formulation for pharmaceutical applications is light mineral oil. This oil is used to produce pharmaceutical formulations useful herein, which include pharmaceutical ingredients, such as FDA-approved dermatological drugs and vitamin supplements of all types, which are soluble at to a reasonable degree in the oil and/or water nanoclusters. Preferred examples of pharmaceutical ingredients that made be included in the inventive compositions and processes include the topical delivery of Vitamins C and E, which may be for example used to prevent or reverse skin damage due to sun exposure or aging. Vitamin C, soluble (clathrated) in the water nanoclusters, stimulates the production of collagen in the skin and functions as an antioxidant along with the antioxidant property of the water nanoclusters. Vitamin E, soluble in the oil, functions along with the water nanoclusters as antioxidant scavenger of cell-damaging free radicals, and the present invention provides for effective delivery thereof to the skin. Additional or alternative preferred pharmaceutical ingredients include FDA-approved transdermally deliverable “classic” drugs such as hormonally active testosterone, progesterone, and estradiol, glycyril trinitrate (e.g., for treatment of angina), hyoscine (e.g., for seasickness), nicotine (e.g., for smoking cessation); prostaglandin E1 (e.g., for treatment of erectile dysfunction); proteins and peptides; DNA and oligonucleotides (e.g., for gene therapy; DNA vaccines).

The types of suitable surfactants include fatty acids, ethoxylates and long chain alcohols. Short chain alcohols are also used as cosurfactants. A preferred surfactant has a polar end (typically a carboxyl COOH group) which attaches. itself to a water cluster. Preferably, the surfactant also has at least one long (preferably 6-20 carbons) linear or branched hydrophobic “tail” that is soluble in the cosmetic oil. The surfactants are preferably present in the up to 50% by weight range.

Preferred fatty acids include hydrolysis products of edible oils, e.g., soybean or Canola oil. These materials consist mainly of oleic and linoleic acid. Purified cuts of these containing larger amounts of these acids can also be used. Fatty acids are examples of anionic surfactants. Anionic surfactants are known to penetrate and interact strongly with skin (P. Morganti et al., J. Appl. Cosmetol. 8, 23, 1990; 12, 25-30, 1994). Most anionic surfactants can induce swelling of the stratum corneum and the viable epidermis (P. Morganti et al., Int. J. Cosmet. Sci. 5, 7, 1983; M. Chvapil and Z. Eckmayer, Int. J. Cosmet. Sci. 7,41-49, 1985). It has been suggested that in conventional cosmetics, the hydrophobic interaction of the alkyl chains with the substrate leaves the negative end group of the surfactant exposed, creating additional anionic sites of the skin membrane (P. Morganti et al., Int. J. Cosmet. Sci. 5, 7, 1983). However, our preferred water clusters in cosmetic formulations bind the negative end group of the surfactant, reducing or eliminating any skin-irritating effects while actually increasing the hydration level of the tissue.

Some cationic surfactants in skin formulations are more irritating to the skin than the anionics and generally would be less suitable for stabilizing water-cluster nanoemulsions.

Nonionic surfactants have the smallest potential for producing skin irritation. In conventional cosmetic microemulsions, they seem to have the ability to partition into the intercellular lipid phases of the stratum corneum, leading to increased “fluidity” in this region. Water-cluster cosmetic nanoemulsions stabilized by nonionic surfactants or a mixture of nonionics and anionics are the preferred compositions.

Ionic surfactants generally have an advantage over nonionic surfactants in being more effective in stabilizing a given amount of water. In addition, they are far more resistant to emulsion breaking at elevated temperature than nonionics. Nonionics maintain themselves at the interface because the polar groups (e. g., —OH) hydrogen bond with water. However, the hydrogen bond is a weak bond (e.g., about 5 Kcal/mol) and becomes less effective as temperature rises above ambient.

Fatty acids are effective detergents but only when at least partially neutralized. Frequently ammonia or organic bases are used to neutralize fatty acids. Ammonia can be an effective neutralizing agent, but is a very weak base and will serve to neutralize only a fraction of the carboxylate, which is also a weak acid.

Amines are effective organic bases. Common amines are the lower alkanol amines, such as monoethanol amine (MEA), isopropanol amine and 2-butanol amine. Also common are the lower alkyl amines. There is a degree of neutralization significantly less than 100% for carboxylic acid surfactants which is optimum for solubilizing the maximum ratio of water to surfactant.

A common nonionic surfactant class useful herein is ethoxylates. Theses are formed by reacting a mole of alcohol or amine with a number of moles of ethylene oxide (EO). The alcohol or amine generally contains a significant sized hydrocarbon group, for example, an akylated phenol or a long chain (C₁₀-C₂₀) alkyl group. Alcohols frequently used are nonyl phenol and lauryl alcohol. The hydrocarbon group serves as the nonpolar section of the molecule. The alcohol can be a can have more than one —OH group and the amine more than one —H, so several ethoxy chains can be present on one molecule. However these multichain ethoxy compounds don't usually function well as surfactants because they do not easily orient at the interface and pack poorly. The balance between hydrophobicity and hydrophylicity is obtained by choosing the hydrocarbon group and the average number of ethylene oxides added. Commonly 3-5 moles of EO are added per mole alcohol or amine.

Another common class of nonionic surfactants useful herein is long chain (C₁₀-C₂₀) alcohols. These are frequently derived from hydrogenation of fatty acids, e.g., myristyl alcohol from myristic acid. Another source is ethylene oligomerization.

Microemulsions may include a “cosurfactant” (e.g., n-pentanol), which is not in itself a surfactant (i.e., a material that can not be used as the sole surfactant, but which may be included to improve the functioning of the material which per se can be used herein as a surfactant). Use of co-solvents is theorized to lower the interfacial tension and reduce dramatically the surfactant requirement. Other co-solvents included n-butanol, n-hexanol, 2 methyl 1-pentanol, 2 methyl 1-hexanol and 2 ethyl 1-hexanol.

One skilled in the art would readily be able to select the amount and type of surfactant to form the desired water clusters, while taking account other considerations (e.g., skin irritation potential) which may be associated with a particular surfactant(s).

The water cluster/surfactant(s) will be present in the oil as a water-in-oil (W/O) emulsion. The W/O emulsions will be comprise of the water clusters (individual or arrays thereof in the forms, shapes and dimensions described above) with surfactants molecules attached thereto. As shown in FIGS. 2 & 3, the single dodecahredral water cluster with fatty-acid surfactant would exist as a W/O emulsion in the cosmetic oil. The water cluster itself is spherical and has a diameter of about 0.8 nanometer (8 Angstroms), with the surfactant molecule extending from the cluster, resulting in a W/O reverse micelle of about 3 nanometers (30 Angstroms) in diameter. As shown in FIGS. 7 & 8, a five-dodecahedral water cluster needle-like array with fatty-acid surfactant would also exist as a W/O emulsion in the cosmetic oil. The water cluster array itself is needle-like and has one dimension of about 0.8 nanometer an a length of about 3 nanometers, with the surfactant molecule linearly clathrated in the needle cavity, resulting in a cylindrically symmetric W/O micelle of about 4 nanometers. (40 Angstroms) in its largest dimension and about 0.8 nanometers (8 Angstroms) in its smallest dimension

Preferred concentrations of water by weight are about 5-50% with the surfactant concentration (typically one surfactant molecule per water cluster) chosen to maximize the presence of water clusters between about 0.5 and 10 nanometers (about 5-100A), and preferably water clusters about 0.8 nm (about 8A) size in the formulation, to minimize separation of water and oil phases prior to application, thereby ensuring long shelf life.

Application of Water Nanoclusters to the Skin

The present invention provides a process for delivery of water nanoclusters through the outmost layer of skin. First, a water nanocluster composition comprising water nanoclusters having diameters between 0.5 and ten nanometers (5-100A) and preferably water clusters of diameter less than one nanometer (10A) and an oil formulation is prepared. The water nanocluster composition is then applied preferably to the outermost layer of human skin.

The skin as a physiological regulator plays a key role in the general metabolism of water in the body. Thus the moisture level of the outermost layer of the skin, the stratum corneum, is critical to maintaining the skin surface healthy and supple. Yet the stratum corneum is believed to be mainly responsible for the rate limiting of skin moisture permeation through the hydrophobic barrier presented by its intercellular lipids (H. Schaefer et al., in Novel Cosmetic Delivery Systems, S. Magdassi and E. Touitou, Eds., Marcel Dekker, New York, 1999, pp. 949).

First-principles quantum-chemistry computations of the electronic structure and low-frequency vibrational modes of water nanoclusters discussed herein, suggest that the permeating clusters will (1) clathrate and deactivate lipid hydrophobes responsible for the stratum corneum hydration barrier, (2) chemically scavenge free radicals that otherwise damage and age epidermal cells, (3) enhance the transdermal delivery of ingredients and (4) be subject to less water evaporation on the skin surface because of the intrinsic stability of the water nanoclusters.

The present invention provides a process and formulation which is capable of providing an effective (1) skin moisturizer, (2) anti-oxidant capable of reducing cell damage and ageing and (3) a mechanism for the delivery of beneficial cosmetic and/or pharmaceutical ingredients to the skin.

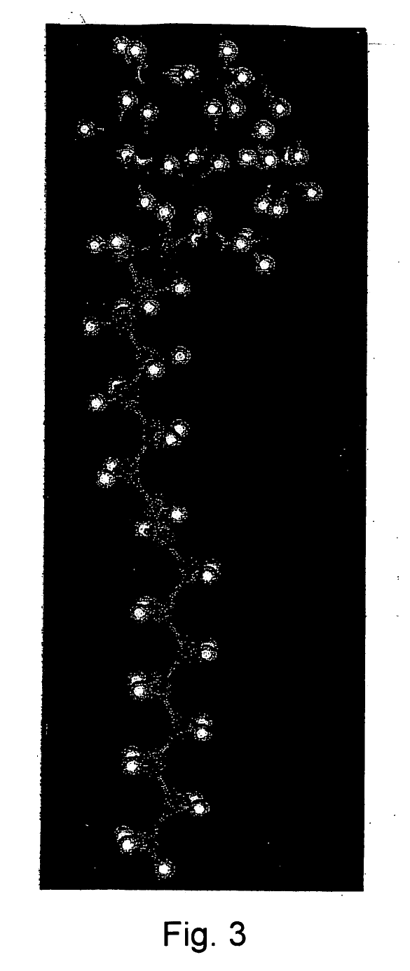

The skin moisturizer benefits are provided due to the present invention's unique capability of effectively overcoming the skin hydration barrier. First, the preferred water clusters of these this invention are less than the 10A (1 nm) size characteristic of the hydrophobic lipid intermolecular spacing and pore diameter of human skin, which enables physical penetration. Second, these water clusters have the unique capability of enclosing or “clathrating” lipid hydrophobes, which thereby counteract the hydrophobic effects of the lipid hydrophobes. This is exemplified in FIG. 3 for a pentagonal dodecahedral water cluster clathrating the end of a typical fatty acid lipid.

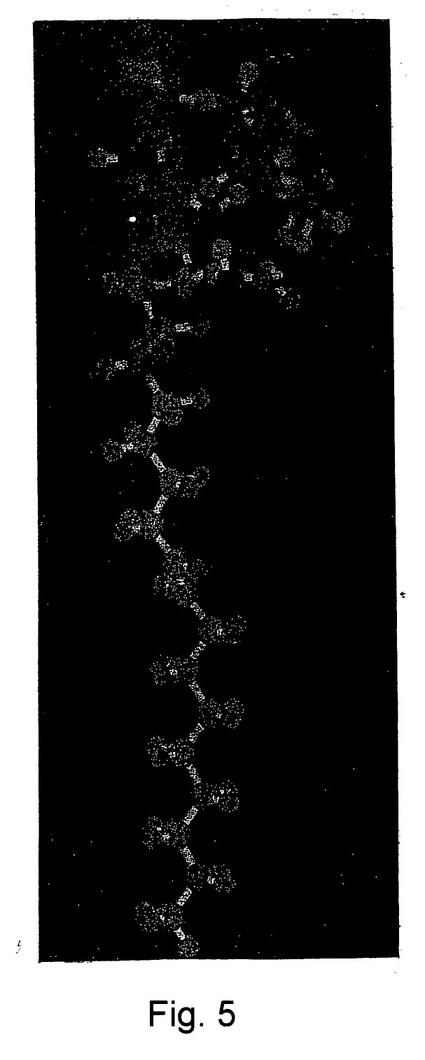

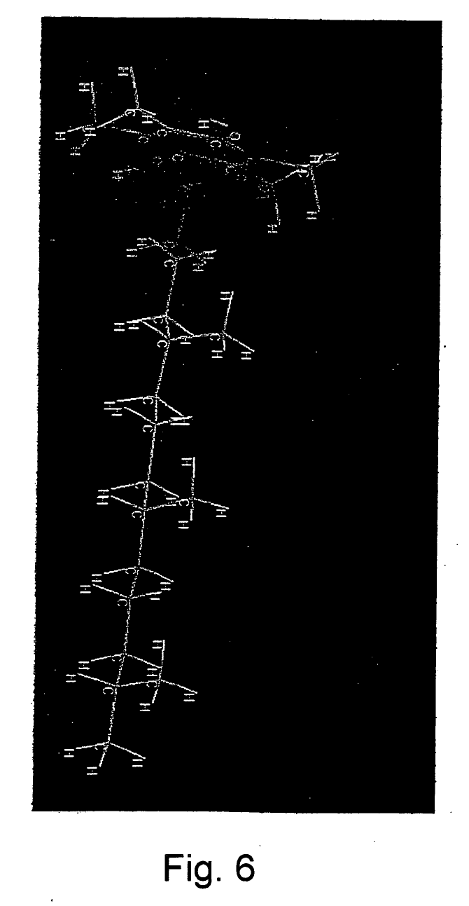

The antioxidant benefits include chemically scavenging free radicals that otherwise damage and age epidermal cells. These benefits are obtained from the functionality of these water clusters after the formulation containing them has been applied to the skin and effectively penetrate the to the outermost layer of human skin. After such penetration has occurred, these water clusters further serve as active antioxidants for scavenging cell-damaging free radicals. Providing anti-oxidarits, such as Vitamin E, to the human body by ingestion and dermal penetration has been a matter of considerable technical and commercial focus. Vitamin E antioxidant function is believed to be associated with its ability to donate electrons to cell-destroying free radicals via the pπ molecular electron orbitals located on the carbon ring moiety at one end of the molecule, as shown in FIG. 6. Without being limited to the theoretical explanation thereof, it is believed that the antioxidant functionality of the water cluster formulations of this invention is generally¹similar to that of Vitamin E but is based upon the electron-donating power of the unique water-cluster surface pπ molecular electron orbitals, coupled with the low-frequency water-cluster breathing vibrational modes through the dynamic Jahn-Teller effect. As shown in FIGS. 5 & 9, the unique water-cluster surface pπ electron-donating molecular orbitals are qualitatively similar to the pπ molecular electron orbitals located on the carbon ring moiety at one end of the Vitamin E molecule shown in FIG. 6.

Individual pentagonal or needle-like arrays of pentagonal dodecahedral clusters like the ones shown in FIGS. 2 & 7 holding an extra electron donated by the surfactant (FIGS. 3 & 10) are potentially powerful antioxidants equal to or better than Vitamin E because of the effectively large reactive cross sections of the cluster surface delocalized oxygen pπ orbitals mapped in FIGS. 5 & 9. As shown in FIGS. 5 & 9, these water clusters can function as electron reservoirs for chemical reactions involving electron transfer to the reacting species. Thus water-cluster hydrated-electron delocalized orbitals, originating on the cluster surface oxygen atoms, can readily overlap with and scavenge cell-damaging free radicals.

Small polyhedral clusters of water molecules, especially quasiplanar and concatenated pentagonal water clusters (e.g. FIGS. 1 & 2), have been experimentally identified as being key to the hydration and stabilization of biomolecules (M. M. Teeter, Proc. Natl. Acad. Sci. 81, 6014. 1984), proteins (T. Baker et al., in Crystallography in Molecular Biology, D. Moras et al., Eds., Plenum, New York, 1985, pp 179-192), DNA (L. A. Lipscomb etal., Biochemistry 33, 3649, 1994), and DNA-drug complexes (S. Neidle, Nature 288, 129, 1980). Such examples indicate the tendency of water pentagons to form closed geometrical structures like the pentagonal dodecahedra shown in FIGS. 1 and 2. It has also been suggested that such water clusters may play a fundamental role in determining biological cell architecture (J. G. Watterson, Molec. And Cell. Biochem. 79, 101, 1988). Approximately 70 percent of the human body is water by weight. Much of that water is believed not to be ordinary bulk liquid, but instead, nanoclustered, restructured water which affects biomolecular processes ranging from protein stability to enzyme activity (J. L. Finney, Water and Aqueous Solutions, G. W. Nelson and J. E. Enderby. Eds., Adam Hilger, Bristol, 1986, pp. 227-244).

### EXAMPLES

### Example 1

A Water Nanocluster/Cosmetic Oil formulation is prepared by mixing the following ingredients to make 1 Kg of formulation.

| Component | Weight Percent |
|---|---|
| Soybean Oil | 50 |
| Water | 25 |
| Surfactant | 20 |
| Surfactant II | 4 |
| Surfactant III | 1 |

The water is deionized. Surfactant I is an ethoxylate with the molecular structure C₈H₁₇ (OCH₂CH₂)₆OH. Surfactant II is a polyglyceryl-oleate. Surfactant III (a cosurfactant) is n-pentanol.

The nanoemulsions are prepared by mixing the soybean oil with Surfactants I and II. Water and Surfactant III are then added simultaneously.

The resultant Water Nanocluster/Cosmetic Oil formulations is a W/O emulsion, with a significant population of stable water nanoclusters in the The water is deionized. Surfactant I is a partially (80%) neutralized (with isopropanol amine) soybean fatty acid. Surfactant II is an ethoxylate with the molecular structure C₈H₁₇ (OCH₂CH₂)ₘOH. Surfactant III (a cosurfactant) is n-pentanol.

The nanoemulsions are prepared by mixing the soybean oil with Surfactants I and II. Water and Surfactant III are then added simultaneously.

### Example 4

A cosmetic oil in which the water is not in the form of nanosized micelles is made as follows:

| Component | Weight Percent |
|---|---|
| Soybean Oil | 73 |
| Water | 25 |
| Surfactant I | 1 |
| Surfactant II | 3 |

The water is deionized. Surfactant I is a polyglyceryl-leate. Surfactant II (a cosurfactant) is n-pentanol. The nanoemulsion is prepared by mixing the soybean oil with Surfactant I. Water and Surfactant II are then added simultaneously.

Three grams of this formulation are placed on a watch glass and this watch glass is placed on a scale. Three grams of the formulation of Example 1 are placed on another watch glass on another scale. Weight losses for each are as follows:

Weight loss, mg.

| Time, hr. | Example 1 | Example 4 |
|---|---|---|
| 1 | 28 | 122 |
| 2 | 62 | 226 |
| 3 | 83 | 307 |

### Example 5

referred size range deliverable to the skin are prepared. The water nanoclusters are in the <2-10 nm nanocluster range as determined by dynamic light scattering and Raman spectroscopy to identify water clusters below 2 nm through their well defined vibrational spectra.

The resultant formulation is applied to the skin, as in any conventional cosmetic application, and penetrates the outmost layer of the skin.

### Example 2

A second formulation is made as follows:

| Component | Weight Percent |
|---|---|
| Soybean Oil | 50 |
| Water | 25 |
| Surfactant I | 12 |
| Surfactant II | 12 |
| Surfactant III | 1 |

The water is deionized. Surfactant I is an ethoxylate with the molecular structure C₈H₁₇(OCH₂CH₂)₆OH. Surfactant II is a partially (50-80%) neutralized (with isopropanol amine) soybean fatty acid. Surfactant III (a cosurfactant) is n-pentanol.

The nanoemulsions are prepared by mixing the soybean oil with Surfactants I and II. Water and Surfactant III are then added simultaneously.

### Example 3

Another cosmetic formulation is formed from the following ingredients:

| Component | Weight Percent |
|---|---|
| Soybean Oil | 50 |
| Water | 25 |
| Surfactant I | 20 |
| Surfactant II | 4 |
| Surfactant III | 1 |

The cosmetic mixtures of Examples 1 and 4 are made up as above. Five (5) grams of each is placed on two 5 cm×5 cm samples of synthetic skin manufactured by Integra Life Sciences Company, under the trade name Integra, which has a water permeability comparable to that of human skin. Five layers of filter paper are placed under each skin sample. Periodically the filter paper samples are weighed. The percent transport of the water through each skin layer is as follows:

| Time hr. | Example 1 | Example 4 |
|---|---|---|
| 2 | 7 | 2 |
| 5 | 22 | 8 |
| 10 | 41 | 14 |

### Example 6

A transdermal Water Nanocluster/Vitamin C/Oil antioxidant formulation is prepared by mixing the following ingredients to make 1 Kg of formulation.

| Component | Weight Percent |
|---|---|
| Light mineral oil | 40 |
| Water | 25 |
| Vitamin C | 10 |
| Surfactant | 20 |
| Surfactant II | 4 |
| Surfactant III | 1 |

The water is deionized. Surfactant I is an ethoxylate with the molecular structure C₈H₁₇ (OCH₂CH₂)₆OH. Surfactant II is a polyglyceryl-oleate. Surfactant III (a cosurfactant) is n-pentanol.

The nanoemulsions are prepared by mixing the mineral oil with Surfactants I and II. Water, Vitamin C, and Surfactant III are then added simultaneously.

The resultant Water Nanocluster/Vitamin C/Oil formulation is a W/O nanoemulsion, with a significant population of stable water nanoclusters clathrating the Vitamin C in the preferred size range deliverable to the skin are prepared. The water nanoclusters are in the <2-10 nm nanocluster range, as determined by dynamic light scattering and Raman spectroscopy to identify water clusters below 2 nm through their well defined vibrational spectra.

The resultant formulation is applied in small amounts to the skin and penetrates the outmost layer of the skin.

### Example 7

A transdermal Water Nanocluster/Oil/Vitamin E antioxidant formulation is prepared by mixing the following ingredients to make 1 Kg of formulation.

| Component | Weight Percent |
|---|---|
| Light mineral oil | 40 |
| Water | 25 |
| Vitamin E | 10 |
| Surfactant | 20 |
| Surfactant II | 4 |
| Surfactant III | 1 |

The water is deionized. Surfactant I is an ethoxylate with the molecular structure C₈H₁₇ (OCH₂CH₂)₆OH. Surfactant II is a polyglyceryl-oleate. Surfactant III (a cosurfactant) is n-pentanol.

The nanoemulsions are prepared by mixing the mineral oil with Surfactants I and II and Vitamin E. Water and Surfactant III are then added simultaneously.

The resultant Water Nanocluster/Oil/Vitamin E formulation is a W/O nanoemulsion, with a significant population of stable water nanoclusters in the preferred size range deliverable to the skin are prepared. The water nanoclusters are in the <2-10 nm nanocluster range, as determined by dynamic light scattering and Raman spectroscopy to identify water clusters below 2 nm through their well defined vibrational spectra.

The resultant formulation is applied in small amounts to the skin and penetrates the outmost layer of the skin.

### Example 8

A transdermal water Nanocluster/Nano Zinc Oxide/Oil antibacterial formulation is prepared by mixing the following ingredients to make 1 Kg of formulation.

| Component | Weight Percent |
|---|---|
| Light mineral oil | 40 |
| Water | 25 |
| Nano Zinc Oxide | 10 |
| Surfactant | 20 |
| Surfactant II | 4 |
| Surfactant III | 1 |

The water should be deionized. Surfactant I is an ethoxylate with the molecular structure C₈H₁₇ (OCH₂CH₂)₆OH. Surfactant II is a polyglyceryl-oleate. Surfactant III (a cosurfactant) is n-pentanol.

Most preferably the water nanocluster compositions of this invention are stable (i.e.; they are thermodynamically stable) in the form of water-in-oil (W/O) nanocluster emulsion for extended periods, most preferably, for months or years after they are formulated). Although an oil and water emulsion can be made by various mixing techniques and/or through the use of other surfactants, such emulsions are typically either oil-in-water (O/W) emulsions (i.e.; not W/O emulsions) and/or are not stable (e.g.; significant phase separation occurs immediately or within hours or several days after preparation). In accordance with the present invention, highly stable (e.g.; which remain stable for 24-36 months) water-in-oil nanocluster emulsion for cosmetic applications are provided through the use of surfactants selected from the group consisting of fatty acid and fatty acid amides, most particularly when the cosmetic oils and the surfactant are mixed prior to the addition of the water, as shown below in Examples 9 and 10.

As discussed hereinabove, a preferred surfactant has a polar end (typically a carboxyl COOH group) which attaches itself to a water cluster and the surfactant also has at least one long (preferably 6-20 carbons) linear or branched hydrophobic “tail” that is soluble in the cosmetic oil. Fatty acid amides are most preferred including the simple fatty acid amides (having the formula R—CO—NH2), which result from the replacement of the hydroxyl of the carboxyl group with an amino group and fatty acid alkanolamides (having the formula R—CO—NH—CH₂₋CH₂—OH), typically derived from fatty acids (e.g.; coconut oil) and alkanolamines. Among the most preferred fatty acid amides are Tallamide diethanolamine (DEA) and Cocamide DEA obtainable from McIntyre Group, Ltd., University Park, Il.60466, under the trade names Mackamide TD and Mackamide C-5, respectively. These surfactants, when used in the preparation of water nanocluster compositions of this invention, by mixing mineral (cosmetic) oil and the surfactant prior to the addition of the water, form water-in-oil nanocluster emulsion form for extended periods which remain stable essentially.

Additional materials such as PPG-3 Myristyl Ether, may also be used to enhance the mixing of the surfactant and the oil. However, the most important mixing benefit is obtained by the order of mixing (i.e.; mixing the cosmetic oil and surfactant prior to the addition of the water components).

As noted above, one skilled in the art would readily be able to select the amount and type of surfactant to form the desired water clusters, while taking account other considerations (e.g.; skin irritation potential) which may be associated with a particular surfactant(s) as well as avoiding the use of other ingredients, which may be unsuitable or limit the intended end-use. For example, although a variety of surfactants are noted in the preparation of nanoemulsions discussed in U.S. Pat. Nos. 5,800,576 and 5,997,590 and suitably form nano-emulsions with the diesel oils and other fuels oils for the combustion-related uses therein, such surfactants may not necessarily form the stable water nanocluster compositions of the cosmetic and pharmaceutical oils in the present invention (because of the inherent differences in these types of oils) and/or the hazardous properties of these oils. Further, although trimethylpentane may have been considered as a potential cosmetic in some applications, due to its hazardous properties, including skin contact hazards, such materials are not considered to cosmetic oils as the term is used herein.

### Example 9

An preferred water nanocluster compositions of this invention is prepared by mixing the following ingredients in the specified approximate weight percentages:

| | |
|---|---|
| Mineral Oil | 65.8% |
| Tallamide DEA | 11.3% |
| Distilled Water | 22.9% |

The mixing procedure involves adding the components in the order indicated above, with the oil/surfactant components premixed with a little stirring prior to the addition of the distilled water. Thick whitish tendrils are formed as the water is added drop wise into the oil/surfactant mixture. After a little stirring and a few seconds time, the final blend clarifies, indicative of the formation of a water-in-oil (W/O) nanoemulsion. The formulation at this point is a pale yellowish liquid of medium viscosity, with a very slight haze. This product is non-irritating to skin and remains a stable nanoemulsion for over 36 months.

Dynamic light-scattering measurements of the nanoemulsions indicate water-micelles between one and six nanometers (10-60 Angstroms) in diameter. Adding more water to the above mixture to a total of approximately 30% water, the mixture becomes whitish, with a tendency to thicken over time. At 40% water, a creamy white emulsion is obtained, similar to a traditional hand lotion in consistency and appearance. Continuing to add water stepwise (about 5% at a time) up to 75% water produces a lotion-like product that is stable. This procedure requires no mechanical mixing whatsoever or application of heat, as is the case for commercial production of cosmetics “pre-mixes”, and therefore is a major cost-saving method of making cosmetic lotions.

### Example 10

Another preferred water nanocluster compositions of this invention is prepared by the same procedure as in EXAMPLE 9, except that a mixture of Tallamide DEA and Cocamide DEA is used as the surfactants, with the percentages being 8.0 wgt % and 3.3 wgt. % respectfully, instead of using 11.30 wgt. % of Tallamide DEA alone. A water-in-oil (W/O) nanoemulsion, which is essentially identical to that of EXAPLE 9 is formed and has essentially identical properties and characteristics.

What is claimed is:

1. A process for delivery of a stable water nanocluster composition through the outermost layer of human skin, comprising the steps of: (1) preparing a water nanocluster composition comprising pentagonal dodecahedral water nanoclusters having at least one dimension thereof being less than about 10 nanometers and an oil formulation in the form of a water-in-oil nano-emulsion, wherein said water nanocluster composition is comprised of at least about 5% by weight water and one or more surfactants selected from the group consisting of one or more fatty acids and fatty acid amides, and said oil formulation is selected form the group consisting of cosmetic and pharmaceutical oils, and (2) applying said water nanocluster composition onto the outermost layer of human skin.

2. The process of claim 1 wherein said water nanocluster composition is comprised of about 5 to 25% by weight of one or more fatty acid amides.

3. The process of claim 2 wherein said water nanocluster composition is comprised of 10-50% by weight water.

4. The process of claim 3 wherein said oil formulation is a cosmetic oil formulation.

5. The process of claim 4 wherein said one or more fatty acid amides t is selected from the group consisting of Tallamide DEA and Cocamide DEA.

6. The process of claim 1 wherein said water nanoclusters in said water nanocluster composition are present together with bulk water but constitute the predominant form of water present in said water nanocluster composition.

7. The process of claim 1 wherein said oil formulation includes pharmaceutical ingredients.

8. The process of claim 1 said water nanocluster composition is prepared by first mixing said oil and surfactant components, and thereafter adding the water component.

9. A stable water-in-oil nano-emulsion composition comprised of (1) about 5 to 50% by weight water containing pentagonal dodecahedral water nanoclusters having at least one dimension of less than about 10 nanometers, (2) about 10 to 90% by weight of an oil formulation, wherein said oil formulation is selected form the group consisting of cosmetic and pharmaceutical oils and (3) about 5 to 50% by weight of one or more fatty acid amide surfactants.

10. The composition of claim 9 wherein said water cluster are in multi-water cluster arrays.

11. The composition of claim 10 wherein said multi-water cluster arrays are needle-like in form, having at least one dimension less than about 1 nanometer and a second dimension greater than about 3 nanometers.

12. The composition of claim 11 wherein said surfactants are clathrated by said water nanoclusters and extend therefrom resulting in reverse micelles of about 3 nanometers in diameter.

13. The composition of claim 12 wherein said surfactants are linearly clathrated in the needle cavity resulting in cylindrically symmetric micelles with a large dimension of about 4 nanometers and a small dimension of about 0.8 nanometers.

14. The composition of claim 9 wherein the said composition is in the form of a gel.

15. The composition of claim 9 wherein the said composition is in the form of a cream.

16. The composition of claim 9 wherein the said composition is in the form of a liquid.

17. The composition of claim 9 wherein the oil formulation is a cosmetic oil formulation selected from the group consisting of soybean, peanut, olive, sesame and paraffin.

18. The composition of claim 17 wherein said cosmetic oil formulation includes one or more additives selected from the group consisting of nutrients, fragrances and sunscreens, which are soluble in the cosmetic oil formulation.

19. The composition of claim 9 wherein said oil formulation includes pharmaceutical ingredients.

20. A process for preparing of water nanocluster compositions suitable for delivery to the outermost layer of human skin, comprising of steps of: (1) mixing one or more surfactants selected from the group consisting of fatty acid and fatty acid amides and an oil formulation selected form the group consisting of cosmetic and pharmaceutical oils and thereafter adding water the mixture thereby preparing a stable water nanocluster composition comprising water nanoclusters having at least one dimension thereof being between about 0.8 and about 10 nanometers and an oil formulation in the form of a water-in-oil nano-emulsion, wherein said water nanocluster composition is comprised of at least about 5% by weight water.

21. The process of claim 20 wherein said one or more surfactants is comprised of about 5 to 25% by weight of fatty acid amides.

22. A stable water-in-oil nano-emulsion composition comprised of:
(1) about 5 to 50% by weight water containing water nanoclusters having at least one dimension between about 0.8 and about 10 nanometers,
(2) about 10 to 90% by weight of an oil formulation, wherein said oil formulation is selected form the group consisting of cosmetic and pharmaceutical oils and
(3) about 5 to 50% by weight of one or more fatty acid amides surfactants.
